(19) United States
(12) Patent Application Publication
(10) Pub. No.: US 2025/0302599 A1
(43) Pub. Date: Oct. 2, 2025

(54) MAMMARY DOSER WITH CARTRIDGES

(71) Applicant: IMAGINUS LIMITED, Auckland (NZ)

(72) Inventors: Robert Holmes, Auckland (NZ); Paul Buckley, Auckland (NZ)

(73) Assignee: IMAGINUS LIMITED, Auckland (NZ)

(21) Appl. No.: 18/618,516

(22) Filed: Mar. 27, 2024

(51) Int. Cl. A61D 1/02; A61M 5/00

(52) U.S. Cl. CPC A61D 1/02; A61M 5/002; A61M 2005/004; A61M 2250/00

(57) ABSTRACT

A mammary-dosing system for infusing a fluid into a teat of a mammal includes a plurality of medicament cartridges and a dosing gun configured to dispense the fluid from each of the medicament cartridges. In accordance with the techniques of this disclosure, each of the medicament cartridges includes: a cartridge body defining an internal cavity configured to retain a fluid; a cannula extending radially upward from an upper surface of the cartridge body and communicatively connected to the internal cavity for dispensing the fluid from the cavity; and a horizontal-alignment device.

### FIELD

The present technology is generally related to animal agriculture. More particularly, the invention relates to prevention of agricultural diseases, such as mastitis.

### BACKGROUND

Mastitis is a significant animal-health problem affecting global dairy production. Dosing a herd of dairy cows to treat or protect against mastitis can be a very labor-intensive exercise. For example, the treatment procedure recommended by Dairy NZ involves cleaning the udder of each cow prior to inoculation, in order to ensure proper hygiene. This process lasts, on average, about 3 minutes per cow. A typical herd of about 400 cows requires over 20 hours of labor. This demand is further accentuated by the fact that at least 4 dosing syringes have to be obtained and manipulated for each cow. In cases where both an antibiotic and a teat sealant are administered to a teat this can mean up to 8 syringes used per cow.

A further issue with treating cows for mastitis is that the inoculation equipment should be scrupulously clean to avoid bacterial contamination. However, many currently available dosing syringes require the user to manually remove a cap each time a cow is dosed, which presents an opportunity for bacteria to contaminate the syringe and infect the COWS.

Improved mammary-dosing apparatuses are disclosed in U.S. Pat. No. 10,136,978 issued to Paul Buckley et al., and in U.S. Pat. No. 10,786,342 issued to Robert Holmes et al., the entire contents of both of which are hereby incorporated in their entirety. As detailed below, the present disclosure improves even further upon these designs.

### SUMMARY

The techniques of this disclosure generally relate to various example features of a mammary-doser and its associated dosing cartridges. In a first example, the present disclosure provides for a mammary-dosing system that includes: a plurality of medicament cartridges, each medicament cartridge comprising: a cartridge body defining an internal cavity configured to retain a fluid; a cannula extending radially upward from an upper surface of the cartridge body and communicatively connected to the internal cavity for dispensing the fluid from the cavity; and a horizontal-alignment device; and a doser gun comprising: a cartridge magazine configured to retain the plurality of medicament cartridges; and a plunger assembly configured to dispense the fluid from an upper-most cartridge of the plurality of medicament cartridges.

In a second example, the present disclosure provides for a method of treating a teat of a mammal, the method including: loading a plurality of medicament cartridges into a cartridge magazine of a dosing gun, wherein each of the plurality of medicament cartridges comprises: a cartridge body defining an internal cavity configured to retain a fluid; a cannula extending radially upward from an upper surface of the cartridge body and communicatively connected to the internal cavity for dispensing the fluid from the cavity; and a horizontal-alignment device; and actuating a plunger assembly of the dosing gun to dispense the fluid from the cannula of an upper-most cartridge of the plurality of medicament cartridges.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A is a cross-sectional side view of an example mammary-dosing system, including a dosing gun and a plurality of interlocking medicament cartridges.

FIG. 1B is a cross-sectional side view of an earlier implementation of the mammary-dosing system of FIG. 1A, highlighting a particular configuration thereof that the system of FIG. 1A is configured to address.

FIG. 2A is a rear-perspective view of a single medicament cartridge of a mammary-dosing system as described herein.

FIG. 2B is a cross-sectional side view of the medicament cartridge of FIG. 2A.

FIG. 2C is a rear-perspective view of a pair of interlocked medicament cartridges.

FIG. 3 is a side view of a distal portion of a plunger assembly of a dosing gun separating the pair of medicament cartridges of FIG. 2C.

FIG. 4A is a rear-perspective view of a stack of four interconnected medicament cartridges with an adhesive label coupled across their proximal ends.

FIG. 4B is a rear-perspective view of a stack of four interconnected medicament cartridges with an adhesive label coupled across their lateral sides.

FIG. 5 is a cross-sectional rear-perspective view of a proximal portion of a mammary-dosing gun, revealing a proximal portion of the plunger assembly housed therein.

FIG. 6A is a perspective view of an example grooved block of the plunger assembly of FIG. 5.

FIG. 6B is a conceptual diagram of the grooved block of FIG. 6A, illustrating an example functionality of the block's two-step guide track.

FIG. 7A is a side view of an example of the mammary-dosing system of FIG. 1A, in which the dosing gun features a cartridge-release tab oriented in a “forward” or “locked” position.

FIG. 7B is a side view of the system of FIG. 7A, in which the cartridge-release tab is oriented in a “rear” or “unlocked” position, enabling manual release of a medicament cartridge from the dosing gun without dispensing the fluid therein.

### DETAILED DESCRIPTION

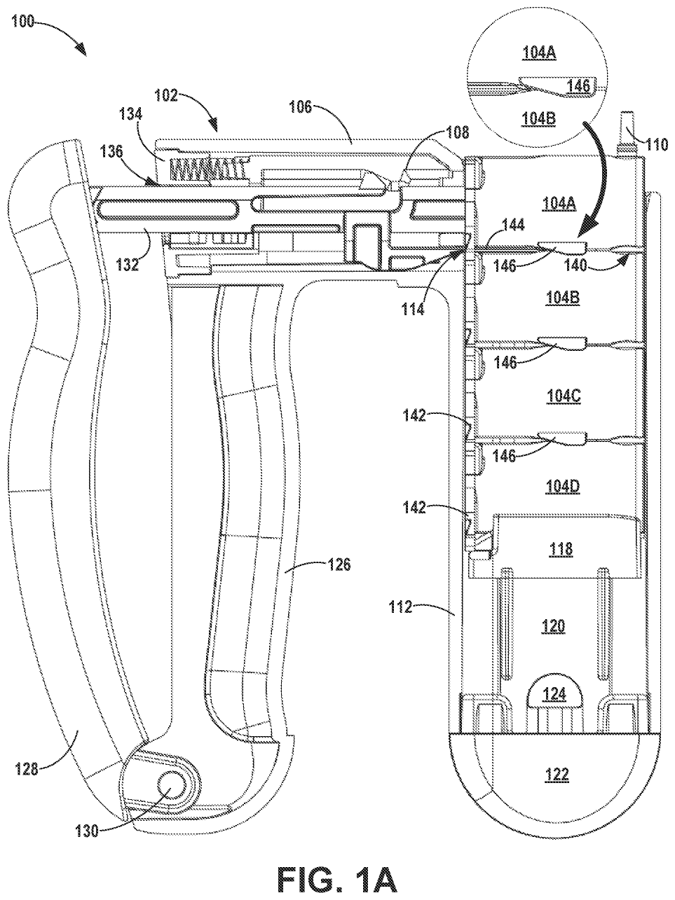

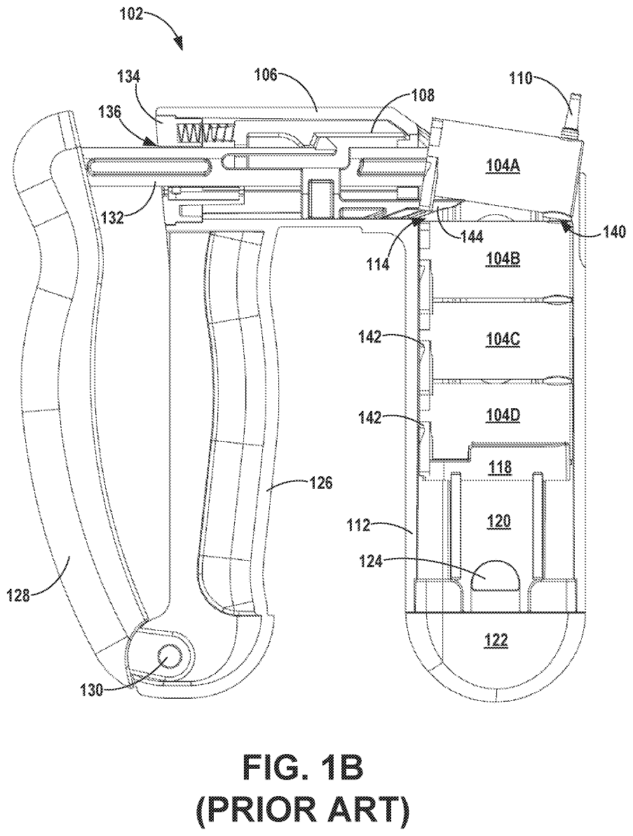

The present disclosure is directed to various example features of a mammary-dosing system, including a plurality of medicament cartridges and a dosing gun configured to dispense a medicament (e.g., a medicine, vaccine, inoculation, anti-venom, antibiotic paste, teat sealant, or any other health-promoting substance) into the teat, udder, or mammary gland of a mammal, such as a cow. Additional details and features of such systems are described in commonly-assigned U.S. Pat. No. 10,136,978 issued to Paul Buckley et al., and in U.S. Pat. No. 10,786,342 issued to Robert Holmes et al., the entire contents of both of which are hereby incorporated in their entirety. FIG. 1A is a cross-sectional side view of an example mammary-dosing system 100, in accordance with some techniques of the present disclosure. Dosing system 100 includes a dosing gun 102 and a plurality of medicament cartridges 104A-104D.

Dosing gun 102 includes a main body 106, which provides structure and a means of operating the invention by hand, and a plunger assembly 108 for engaging with the cartridges 104 one-by-one, and distally advancing the piston of the cartridges 104 to dispense a medicament (e.g., vaccine) retained within the cartridge 104 upward and outward through the cannula 110.

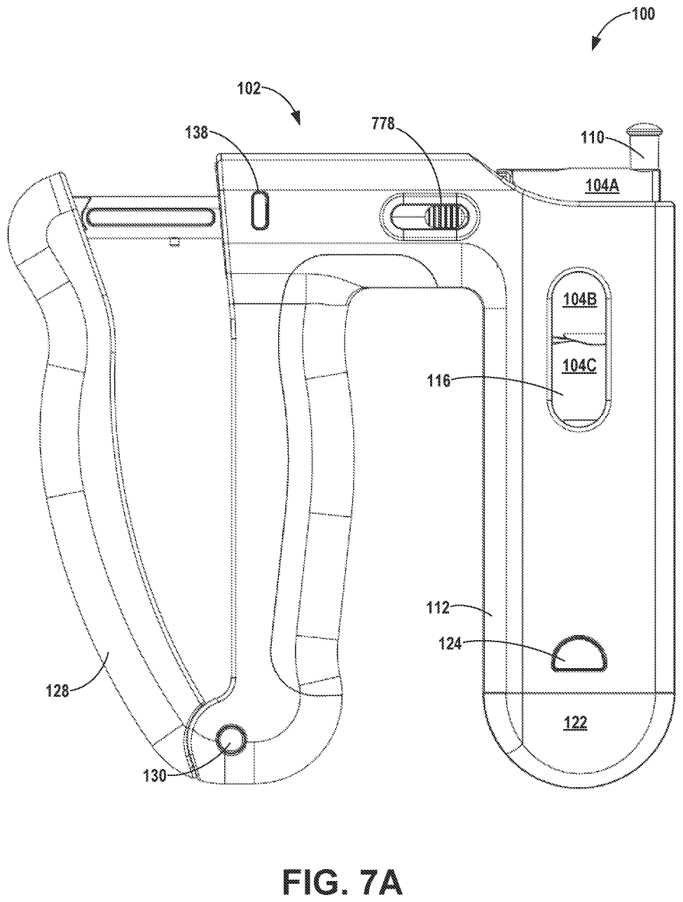

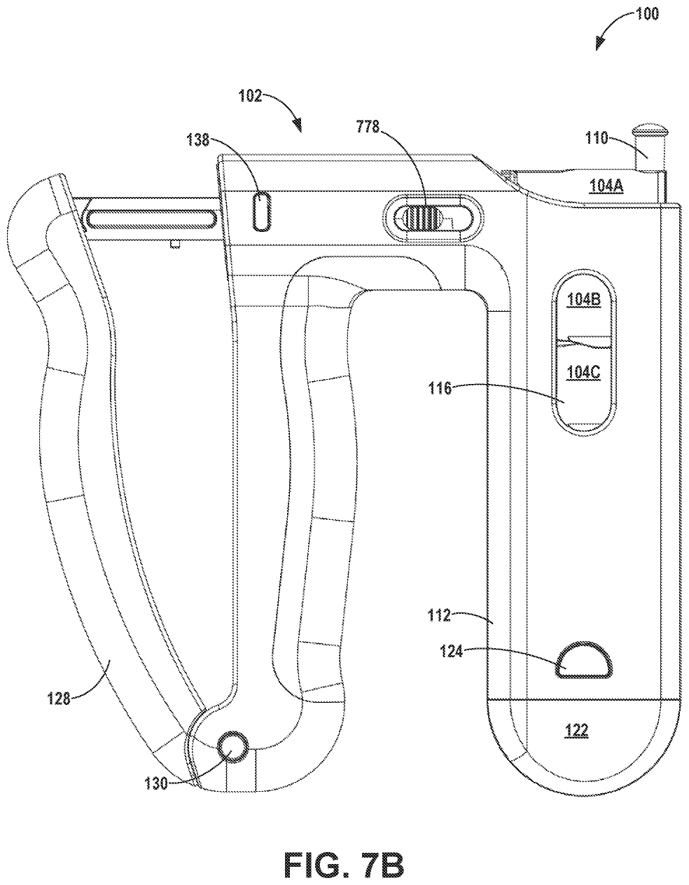

A front column of the main body 106 serves as a cartridge magazine 112 that stores the medicament cartridges 104 and feeds them upward into a breach 114 one after the other. On one or both lateral sides of the magazine 112 is a vertically oriented viewing aperture 116 (shown in FIGS. 7A and 7B) that enables a user to visually determine the number of cartridges 104 remaining inside the magazine 112. Inside the magazine 112, the stack of cartridges 104 is cradled from below by a follower 118, which itself is urged upwards by a magazine spring 120. The magazine spring 120 uses a magazine cap 122 as a support/foundation, while the cap 122 is configured to clip in through a pair of clip apertures 124 on both sides of the magazine 112, thus securing the magazine cap 122 to the magazine 112 and sealing the open-ended bottom of the magazine 112 from dirt and debris.

The rear column of the body 106 forms a handle 126, by which the user holds and operates the dispenser gun 102. The handle 126 supports a lever-style trigger mechanism 128, pivoting from and attaching by clip-in at a pair of handle apertures 130. The handle 126 and trigger 128 are ergonomically shaped with comfortable gripping surfaces. The trigger mechanism 128 is configured to push a plunger shaft 132 when pushed forward as the user's hand squeezes the trigger 128 and handle 126 together. A V-shaped trigger spring (not shown) running through the handle 126 and trigger 128 returns the trigger 128 and the plunger shaft 132, which is connected to the trigger 128, to a retracted position when the user releases the squeeze of their hand.

A handle cap 134 defining a proximal aperture 136 enables the plunger shaft 132 to slide in and out of the body 106 in a stabilized manner. The handle cap 134 is configured to clip into a pair of body apertures 138 (shown in FIGS. 7A and 7B) on either side of the main body 106, thus sealing the section of the body 106 enclosing the plunger assembly 108 from the rear.

Adjacent medicament cartridges 104 are removably coupled together by (1) the interlocking fit of a lower-cartridge cannula 110 upward into a counterpart aperture 140 defined by a lower (or “ventral”) surface of the adjacent cartridge above, and (2) at the rear, by a series of interlocking tabs 142. Rear interlocking tabs 142 hold adjacent cartridges together until separated by a blade portion 144 of the plunger assembly 108. Meanwhile, a head portion of the plunger 108 extends distally forward against the piston on the backside of cartridge 104A, causing the medicament to be dispensed outward through the cannula 110.

In earlier implementations of system 100, an example of which is reproduced in FIG. 1B for reference, the wedge-shaped tip of the plunger blade 144 was configured to extend horizontally between cartridges 104A and 104B, which would first separate the rear/proximal interlock 142 by pushing the lower cartridge 104B downward, before advancing forward even further and subsequently separating the front/distal cannula interlock 110/140. In some such examples, because the plunger blade 144 incrementally raised the back/proximal end of cartridge 104A prior to the separation of the front cannula interlock 110/140, cartridge 104A could assume the configuration depicted in FIG. 1B, in which the front end of cartridge 104A is angled slightly downward along a proximal-to-distal direction. This downward-angled configuration of the upper-most cartridge 104A may be undesirable for some users, who would prefer a more-precise vertical alignment of cannula 110 relative to the teat of the recipient mammal.

Accordingly, additional features of system 100 of FIG. 1A, as detailed further throughout the disclosure below, are specifically designed to help maintain a precise horizontal alignment of the upper cartridge 104A during separation from the cartridge 104B below, and thus, maintain a precise vertical alignment of the cannula 110 extending vertically upward therefrom. Two such features include a more-robust proximal interlocking mechanism 142, and a pair of wedge-shaped ventral fins 146.

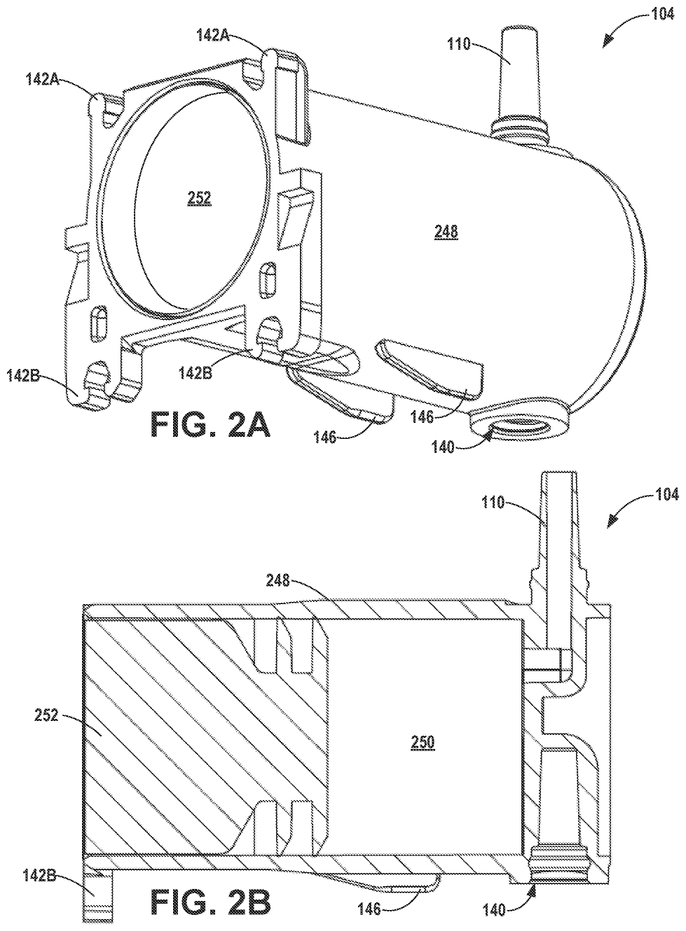

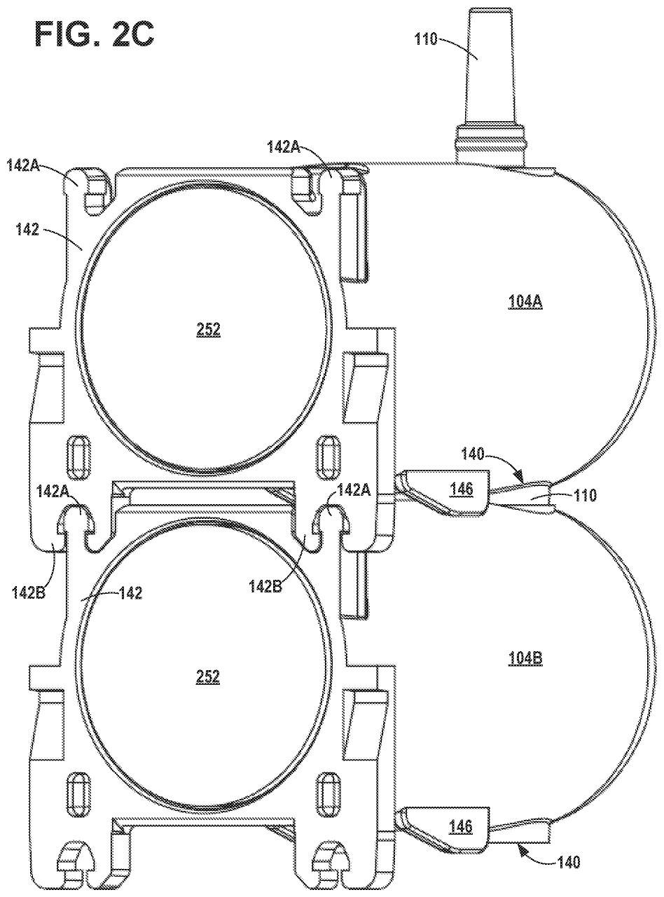

For instance, FIGS. 2A and 2B illustrate an example medicament cartridge 104 of the mammary-dosing system 100 of FIG. 1A. As shown in FIGS. 2A and 2B, cartridge 104 includes a generally cylindrical body 248 defining an internal cavity 250 configured to store a medicament, such as a vaccine, medicine, or other health-promoting substance. When the head of a plunger assembly extends distally forward against the rear piston 252, which then compresses distally forward into cavity 250, the medicament is ejected vertically outward through cannula 110 and onto and/or into the teat of a mammal.

Dosing cartridge 104 further includes a proximal or rear interlocking mechanism 142 configured to removably couple adjacent cartridges together. In general, proximal interlocking mechanism 142 of the present disclosure is configured to be more robust than its counterpart in prior examples, such as the example shown in FIG. 1B. Specifically, as best depicted in FIGS. 2A and 2C, proximal interlocking mechanism 142 includes a generally “H”-shaped structure that surrounds the entire proximal circumference of cartridge body 248. H-shaped interlocking mechanism 142 includes a pair of protrusions or tabs 142A extending vertically upward from an upper portion of the structure, and a counterpart pair of hooked, inverted-U-shaped claws 142B extending downward from a lower portion of the structure. During use, the lower claws 142B on an upper cartridge 104A are configured to grab onto and retain a counterpart pair of upper tabs 142A on an adjacent lower cartridge 104B.

In accordance with the techniques of this disclosure, medicament cartridge 104 includes a feature to help the cartridge remain substantially parallel to the axis of the plunger (i.e., to remain substantially horizontal) while using the dosing gun to dispense medicament from the cartridge. For instance, as shown in FIGS. 2A-2C, each dosing cartridge 104 includes a pair of wedge-shaped fins 146 on an underside or ventral side of the cylindrical body 248. Wedge-shaped fins 146 help maintain the relative alignment between adjacent cartridges 104 while the plunger blade 144 of the dosing gun disengages the proximal locking tabs 142A, 142B therebetween.

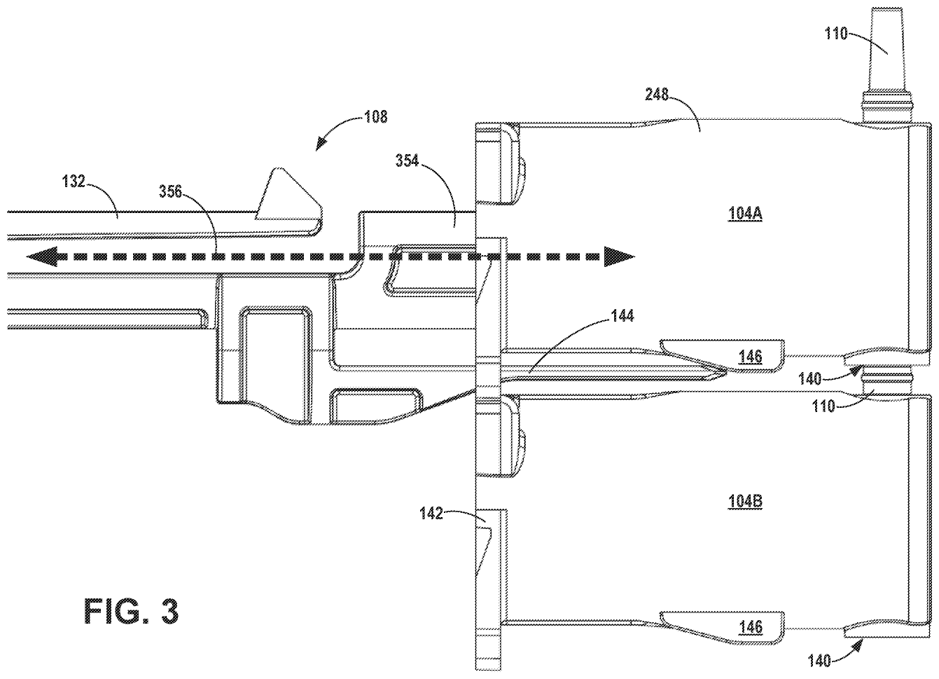

An example of this mechanism is shown in FIG. 3, which illustrates the plunger assembly 108 of a mammary-dosing gun engaged with the pair of medicament cartridges 104A, 104B of FIG. 2C. Specifically, the plunger blade 144 extends distally forward between the two cartridges 104A, 104B until it reaches the wedge-shaped fins 146, at which point the blade 144 slides along the angled lower surface of each of the fins 146 and wedges the two cartridges 104 apart along the vertical direction. Specifically, the addition of ventral fins 146 ensures that the proximal interlock 142 and the distal cannula interlock 110/140 both disengage simultaneously, thereby preventing the upper cartridge 104A from swiveling downward before the proximal interlock 110/140 can disengage. In some examples, a longitudinal distance between the distal tip of the plunger blade 144 and a front end of the plunger head 354 can be varied to adjust a time delay in between the separation of the cartridges 104 and the ejection of the upper cartridge 104A from the dosing gun.

In some examples, but not all examples, due to the mounting location of the wedge-shaped fins 146 on the ventral underside of each cartridge 104, the upper cartridge 104A remains substantially parallel to the horizontal axis 356 of the plunger assembly 108, while the plunger blade 144 may deflect slightly downward while dislodging the upper cartridge 104A. Additionally or alternatively, wedge-shaped fins 146 (or other suitable alignment guides) can be mounted to an upper (or “dorsal”) surface of each cartridge 104. In some such examples, the plunger blade 144 may remain substantially linear while dislodging the upper cartridge 104A.

In some examples, adjacent cartridges 104 can be removably coupled by just a single interlocking mechanism, e.g., positioned centrally along the cylindrical bodies 248 of the cartridges 104, such that the plunger blade 144 separates the cartridges 104 while the upper cartridge 104A remains substantially parallel to the longitudinal axis 356 of the plunger assembly 108. In some such examples, the front/distal cannula interface 110/140 functions as a protective cover or seal for the lower cannula tip, but not as a removable interlocking mechanism for the cartridges.

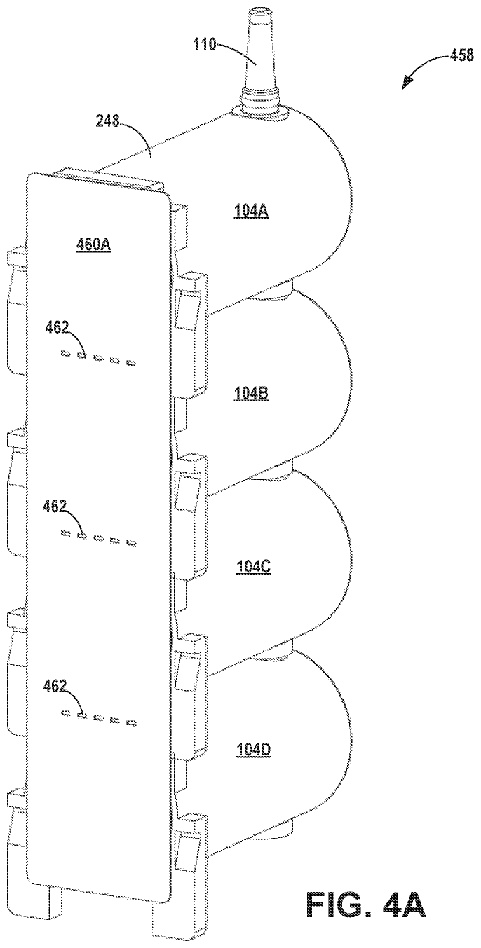

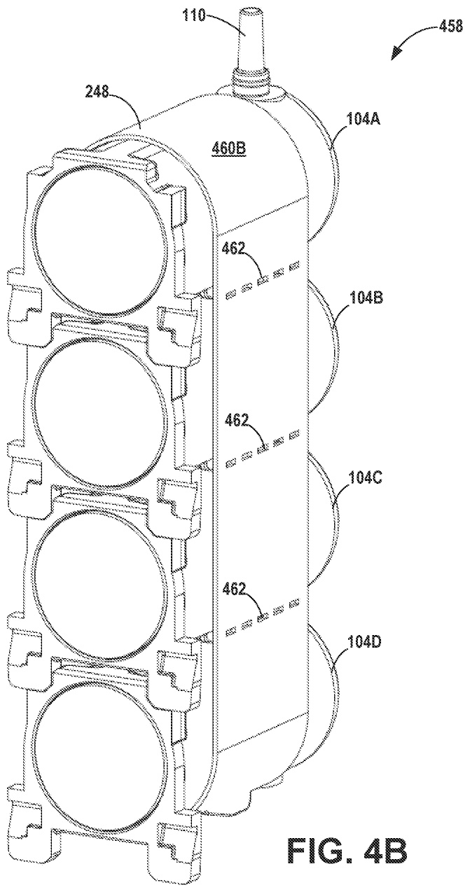

FIG. 4A is a rear-perspective view of an interconnected stack 458 of four medicament cartridges 104A-104D. In addition to, or instead of, proximal interlocking tabs 142, stack 458 includes an elongated label 460A adhered (e.g., glued, heat stacked, or ultrasonically welded) to the proximal/rear end of the cartridges 104 to temporarily retain the cartridges 104 together within the stack 458. In some instances, label 460A is configured to help maintain the horizontal alignment of the upper-most cartridge 104A until separated by the plunger blade 144. Label 460A can include, for instance, a paper-based label that is easily pierced by the plunger blade 144. Optionally, label 460A can include printed information such as user instructions, withholding periods, manufacturing batch coding, or the like. In some such examples, the label 460A can include a series of perforations 462 between adjacent cartridges to facilitate splitting of the label 460A by the plunger blade 144 while dislodging the upper-most cartridge 104A.

FIG. 4B is a rear-perspective view of another example of the interconnected stack 458 of four medicament cartridges 104A-104D of FIG. 4A. In addition to, or instead of, proximal interlocking tabs 142 and/or proximal label 460A, stack 458 includes an elongated label 460B adhered around the lateral sides of the cartridges 104 to temporarily retain the cartridges 104 together within the stack 458. In some instances, label 460A is configured to help maintain the horizontal alignment of the upper-most cartridge 104A until separated by the plunger blade 144. Label 460B can include, for instance, a paper-based label that easily self-separates when the plunger blade 144 applies a vertical expansion force in between cartridges 104A and 104B. Optionally, label 460B can include printed information such as user instructions, withholding periods, manufacturing batch coding, or the like. In some such examples, the label 460B can include a series of perforations 462 between adjacent cartridges to facilitate separation of the label 460B when the plunger blade 144 dislodges the upper-most cartridge 104A.

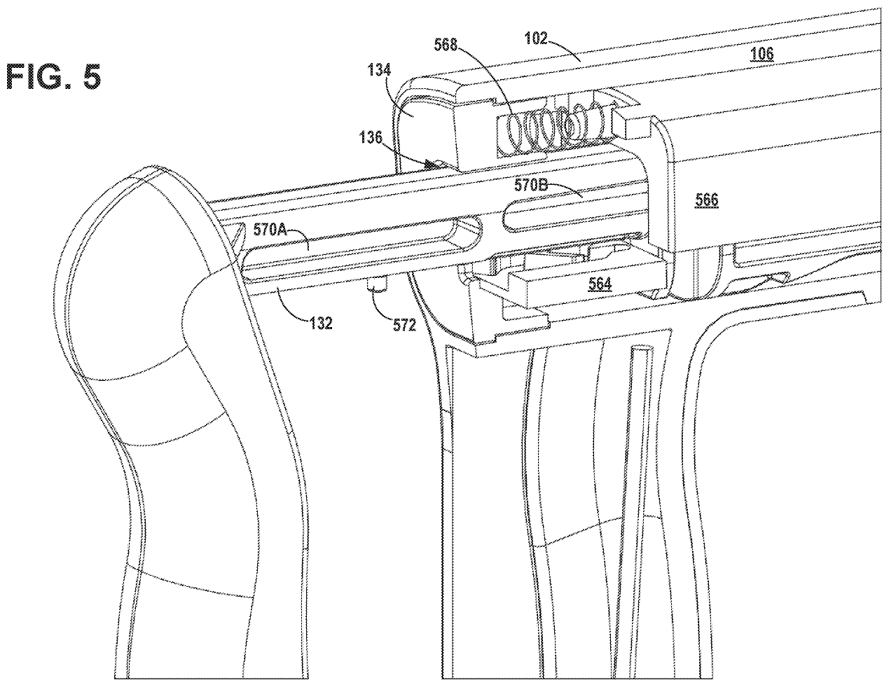

FIG. 5 is a cross-sectional perspective view of a proximal portion of the mammary-dosing gun 102, revealing a proximal portion of the plunger assembly 108 retained therein. In the example of FIG. 5, the plunger assembly 108 of the dispenser gun 102 includes: an elongated shaft 132, a grooved block 564, a slide 566, and a slide spring 568. In some examples, but not all examples, plunger shaft 132 defines a single elongated slots 570, for instance, as an artifact of the manufacturing process, but otherwise not affecting the functionality of the device.

As the plunger shaft 132 moves proximally and distally through handle-cap aperture 136, a cylindrically shaped knob 572, about 2 mm in length, on the shaft's ventral surface is guided through a set of tracks defined by the grooved block 564, which is positioned directly under the knob 572 within the body 106 of the dispenser gun 102. The block 564 is able to move laterally (i.e., side-to-side) within the handle cap 134 as plunger shaft 132 moves proximally and distally, thus enabling knob 572 to navigate the tracks of the block 564.

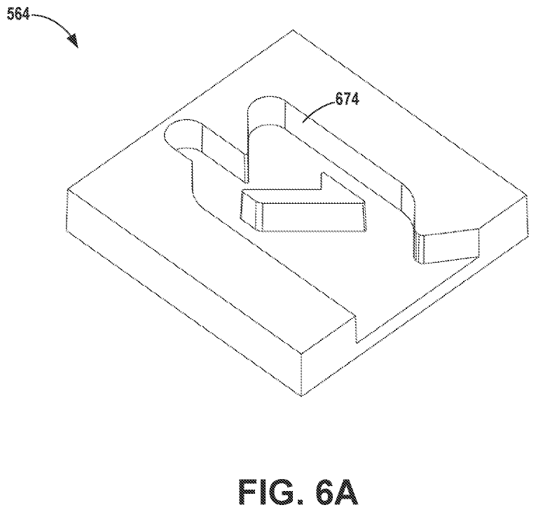

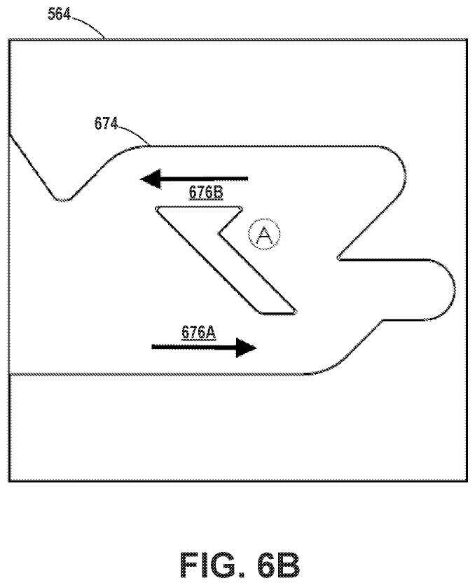

As illustrated further in FIGS. 6A and 6B, the grooved block 564 defines a two-step guide-track 674, which is configured to prevent the dosing gun 102 from automatically ejecting a medicament cartridge 104 until after two consecutive manual inputs (i.e., trigger squeezes) are received from a user. The two consecutive trigger squeezes cause the plunger knob 572 to advance through both portions 676A, 676B, respectively, of the two-step guide track 674.

For instance, in some examples, dosing gun 102 can be configured to operate according to a standard, single-input action mechanism. In such examples, the user squeezes the rear trigger 128 forward toward the handle 126, thereby advancing the plunger assembly 108 forward to dispense fluid from the cannula 110 of cartridge 104A. When the user releases their grip, the internal spring returns the trigger 128 back to its proximal position and the dosing gun 102 automatically ejects the upper cartridge 104A.

In other examples, dosing gun 102 can be configured operate according to a double-input action mechanism. In such examples, the user squeezes the rear trigger 128 forward toward the handle 126 to cause the plunger 108 to dispense medicament from the cannula 110 of upper cartridge 104A. While the trigger 128 advances forward, plunger knob 572 advances forward through first guide-track portion 676A of grooved block 564. Upon releasing this first squeeze, the trigger 128 then returns only slightly backward before the plunger knob 572 catches at resting point “A” in FIG. 6B, at which point, upper cartridge 104A has not yet ejected from the dosing gun 102. Only after an additional, secondary manual compression of the trigger 128 does the trigger 128 move forward again, pushing the plunger 108 to its forward-most position to eject the upper cartridge 104A from the breach 114, before fully returning to its proximal position under spring force. This secondary manual compression-and-release causes plunger knob 572 to travel along second guide-track portion 676B within grooved block 664, thereby ejecting the cartridge 104A and resetting the trigger mechanism 128.

As an illustrative example, one advantage of not immediately ejecting the upper cartridge 104A is to keep the cannula 110 of the lower cartridge 104B protected from external sources of contamination while housed within the upper cartridge 104A, until the technician is ready to dose the next teat. This gives the technician time to clean the next teat. Once the next teat has been sufficiently sterilized, a simple squeeze-and-release of the trigger handle 128 releases the spent cartridge 104A to expose the new clean cannula 110.

FIGS. 7A and 7B are side views of an example of the mammary-dosing system 100 of FIG. 1A, in which the dosing gun 102 includes a cartridge-release tab 778. In FIG. 7A, the cartridge-release tab 778 is oriented in a “forward” or “locked” position. By contrast, in FIG. 7B, the cartridge-release tab 778 is oriented in a “rear” or “unlocked” position, enabling manual release of a medicament cartridge 104A from the dosing gun 102 without dispensing the fluid contained therein.

Medicament cartridges 104 may be provided in the form of an interlocked stack having a predetermined number of cartridges (e.g., a stack 458 of four interlocked cartridges 104A-104D, as in the example of FIGS. 4A and 4B). In some applications, after loading the stack 458 of cartridges 104 into the magazine 112, the user may discover that not all of the cartridges 104 within the stack 458 are required.

For instance, in one example scenario, the user may approach a “blind-teat” cow awaiting treatment. Blind-teat cows have at least one teat that is not fully developed or properly functioning. In this case, only three of the blind-teat cow's four teats will need to be treated; thus, only three of the four cartridges 104 in the stack are required. Note that this example is illustrative only-in other examples of dosing system 100, the cartridge stack may initially include more than four cartridges 104, or fewer than four cartridges 104. In such cases, the user can retract the cartridge-release tab 778 in a proximal direction (i.e., to the left, from the perspective shown in FIGS. 7A and 7B). Once the cartridge-release tab 778 is in the retracted configuration shown in FIG. 7B, the corresponding internal mechanism is thereby unlocked, enabling the user to eject one or more unused cartridges 104 from the magazine 112.

All the examples shown and described above are designed primarily for injecting a health-promoting material into a mammal's teat canal. It is envisaged that with minor variations, the dosing gun 102 and/or cartridges 104 and apparatus could be used to inject substances into other cavities, and thus, such variations in usage should not be considered to be outside the scope of the present disclosure.

In the examples explicitly shown and described, the dispenser gun 102 is configured to dispense four cartridges 104 prior to reloading the magazine 112. It is envisaged that variations of the gun 102 could be adapted to dispense fewer or more cartridges 104, as desired.

In summary, it will be understood that embodiments of the present disclosure enable a dosing system that includes a doser gun and one or more cartridges, whereby the system: (1) facilitates a reduction in the total volume of plastic packaging used to store the formulations, i.e. because one gun 102 may have multiple cartridges 104, while still ensuring each dose is still in its own separate package; (2) is simple to load, use and operate with a very comfortable ergonomic design; (3) does not require the user to undertake secondary actions to remove a cap of each cartridge; (4) substantially maintains a horizontal alignment of an upper-most cartridge 104A while dispensing the substance therein; and/or (5) effectively interlocks multiple cartridges into a convenient stack 458 until iteratively expended and ejected by the dosing gun 102.

The embodiments above are chosen, described, and illustrated so that persons skilled in the art will be able to understand the invention and the manner and process of making and using it. The descriptions and the accompanying drawings should be interpreted in the illustrative and not the exhaustive or limited sense. The invention is not intended to be limited to the exact forms disclosed. While the application attempts to disclose all of the embodiments of the invention that are reasonably foreseeable, there may be unforeseeable insubstantial modifications that remain as equivalents. It should be understood by persons skilled in the art that there may be other embodiments than those disclosed which fall within the scope of the invention as defined by the claims. Where a claim, if any, is expressed as a means or step for performing a specified function it is intended that such claim be construed to cover the corresponding structure, material, or acts described in the specification and equivalents thereof, including both structural equivalents and equivalent structures, material-based equivalents and equivalent materials, and act-based equivalents and equivalent acts.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a mammary-dosing device and/or associated medicament cartridges.

What is claimed is:

1. A mammary-dosing system comprising:
a plurality of medicament cartridges, each medicament cartridge comprising:
a cartridge body defining an internal cavity configured to retain a fluid;
a cannula extending radially upward from an upper surface of the cartridge body and communicatively connected to the internal cavity for dispensing the fluid from the cavity; and
a horizontal-alignment device; and
a doser gun comprising:
a cartridge magazine configured to retain the plurality of medicament cartridges; and
a plunger assembly configured to dispense the fluid from an upper-most cartridge of the plurality of medicament cartridges.

2. The mammary-dosing system of claim 1, wherein the horizontal-alignment device extends outward from the cartridge body.

3. The mammary-dosing system of claim 2, wherein the horizontal-alignment device comprises a pair of wedge-shaped tabs coupled to a lower surface of the cartridge body.

4. The mammary-dosing system of claim 1, wherein each medicament cartridge further comprises an interlocking mechanism disposed at a proximal portion of the cartridge body, the interlocking mechanism configured to removably couple adjacent cartridges into a vertical stack.

5. The mammary-dosing system of claim 4, wherein the interlocking mechanism comprises a lower pair of inverted-U-shaped claws, and counterpart pair of upper tabs.

6. The mammary-dosing system of claim 4, wherein the interlocking mechanism extends fully around a circumference of the cartridge body.

7. The mammary-dosing system of claim 1, wherein the horizontal-alignment device comprises an adhesive label coupled across a proximal end of each of the plurality of medicament cartridges.

8. The mammary-dosing system of claim 7, wherein the adhesive label comprises a horizontal perforation line between each adjacent pair of cartridges of the plurality of medicament cartridges.

9. The mammary-dosing system of claim 1, wherein the horizontal-alignment device comprises a label extending around lateral sides of the plurality of medicament cartridges.

10. The mammary-dosing system of claim 1, wherein the plunger assembly comprises:
a plunger head configured to extend distally into the cartridge body to eject the fluid from the cannula;
a wedge-shaped blade configured to separate an upper-most cartridge of the plurality of medicament cartridges from an adjacent lower cartridge of the plurality of medicament cartridges;
an elongated shaft defining a longitudinal axis;
a slide disposed overtop of the plunger head and in sliding contact with the plunger head; and
a spring disposed between a distal end of the slide and an interior surface of a main body of the doser gun.

11. The mammary-dosing system of claim 10, wherein a distal-most tip of the wedge-shaped blade extends distally past a leading edge of the plunger head.

12. The mammary-dosing system of claim 10, wherein the plunger assembly further comprises:
a plunger knob extending downward from the elongated shaft; and
a grooved block defining a guide track configured to receive the plunger knob.

13. The mammary-dosing system of claim 12, wherein the guide track comprises a first track portion, an intermediate resting point, and a second track portion, wherein a first manual actuation of the dosing gun causes the plunger knob to travel through the first track portion and catch at the intermediate resting point to dispense the fluid from the cannula of an upper-most cartridge of the plurality of medicament cartridges, and wherein a second manual actuation of the dosing gun causes the plunger knob to travel through the second track portion to eject the upper-most cartridge from the dosing gun.

14. The mammary-dosing system of claim 1, wherein the dosing gun further comprises a release tab enabling a user to eject, from the magazine, an upper-most cartridge of the plurality of cartridges without dispensing the fluid from the cannula.

15. The mammary-dosing system of claim 1, wherein the system enables a user to administer a prophylactic or therapeutic mastitis product to a teat of a mammal, wherein the cannula is configured to engage the teat of the mammal.

16. The mammary-dosing system of claim 1, wherein the dosing gun further comprises:
an ergonomic handle;
a trigger having a lower end hingeably connected to a bottom portion of the ergonomic handle, and an upper end coupled to a proximal end of the plunger assembly; and
a V-shaped spring extending through the ergonomic handle and the trigger, wherein the V-shaped spring is configured to bias the trigger into a retracted configuration.

17. The mammary-dosing system of claim 1, wherein the dosing gun further comprises a cartridge cap configured to removably couple to a bottom end of the cartridge magazine.

18. The mammary-dosing system of claim 1, wherein the cartridge magazine comprises a transparent window for viewing the plurality of medicament cartridges housed withing the cartridge magazine.

19. A method comprising:
loading a plurality of medicament cartridges into a cartridge magazine of a dosing gun, wherein each of the plurality of medicament cartridges comprises:
a cartridge body defining an internal cavity configured to retain a fluid;
a cannula extending radially upward from an upper surface of the cartridge body and communicatively connected to the internal cavity for dispensing the fluid from the cavity; and
a horizontal-alignment device extending outward from the cartridge body; and
actuating a plunger assembly of the dosing gun to dispense the fluid from the cannula of an upper-most cartridge of the plurality of medicament cartridges.

20. The method of claim 19,
wherein actuating the plunger assembly comprises squeezing a trigger of the dosing gun a first time to dispense the fluid from the cannula of the upper-most cartridge; and
wherein the method further comprises squeezing the trigger of the dosing gun a second time to eject the upper-most cartridge from the cartridge magazine.
